(19) United States
(12) Patent Application Publication
(10) Pub. No.: US 2007/0102501 A1
(43) Pub. Date: May 10, 2007

(54) Device and methods for counting, timing, recording, and charting fetal movement frequency

(76) Inventor: Diep Mong Nguyen, Manhattan Beach, CA (US)

Correspondence Address: BUCHANAN, INGERSOLL & ROONEY PC, POST OFFICE BOX 1404, ALEXANDRIA, VA, 22313-1404, US

(21) Appl. No.: 11/219,405

(22) Filed: Sep. 2, 2005

(51) Int. Cl. G01G 23/18; A61B 8/00

(52) U.S. Cl. 235/1 R; 600/453

(57) ABSTRACT

Methods and a device for charting fetal movements. The device may record 10 fetal movements in a session, wherein each movement is recorded by pressing a button on the device. The device may display an additional footprint icon each time the button is pressed, wherein the user can count the recorded movements during the session. The device may record the elapsed time (KickTime) for each session and store up to 10 KickTimes so that the user can review the frequency of the movements from one sequence to the next. The device may also have a sound recording and playback function, where user may record the fetal heartbeat for playback. The device may store and update the weeks and days of the mother's pregnancy progress. The device may also calculate the countdown days-to-go and display the countdown if the countdown is less than 100.

### FIELD OF THE INVENTION

The present invention relates to fetal health monitoring, more particularly, to an electronic device for helping a pregnant woman count and chart fetal movements.

### BACKGROUND OF THE INVENTION

Perceived fetal movement by a pregnant woman is regarded as an excellent indication of fetal well-being. Fetal movement serves as an indirect measure of the central nervous system integrity and function. Although fetal movement can be documented by ultrasound as early as 7-8 weeks of pregnancy, the first fetal movement or “flutter” is not usually felt by the mother until the 16th week (for women who have delivered a baby) to the 20th week (for women pregnant for the first time). Movements generally increase in strength and frequency through pregnancy, particularly at night, and when the woman is at rest. At the end of pregnancy (36 weeks and beyond), there is normally a slow change in movements, with fewer violent kicks and more rolling and stretching fetal movements.

Maternal counting and tracking of fetal movements is called kick count. It has been accepted as a cheap, simple, valuable, effective, reliable and harmless screening of fetal well-being in both low and high risk pregnancies. For example, a sudden decrease in fetal movements may indicate potential problems and may need further evaluation of fetal well-being. As shown at http://www.MOMStudy.com, the International MOMSTUDY, currently with more than 3000 participants, has found that more than half of stillbirth mothers (nearly three quarter in third trimester unexplained stillbirths) reported their first reason to believe their baby not doing well is a reduction in fetal activity. Half of the affected mothers have perceived a gradual reduction of fetal movement several days before.

Several formal protocols for kick count have been developed over the years. However, there is no single accepted protocol. For example, Pearson and Weaver's study (“British Medical Journal,” Vol. 1, pp. 1305-1307, May 29, 1976) suggested counting 10 fetal movements daily and reporting if there are less than 10 movements in 12 hours. Sadovsky et al. (“Obstetrics and Gynecology,” Vol. 50, No. 1, pp. 49-55, July 1977) suggested counting fetal movements for 30 minutes to one hour, three times a day, and if there are less than 3 movements in one hour, the count is continued for 6-12 hours and reporting if there are less than 10 movements in 12 hours, while Neldam's study (“The Lancet,” pp. 1222-1224, Jun. 7, 1980,) suggested counting fetal movement for 2 hours after a meal, 3 times a week and reporting if there are less than 3 movements per hour. In Moore and Piacquadio's study (“American Journal of Obstetrics and Gynecology,” Vol. 160, pp. 1075-1080, May 1989), the fetal mortality rate significantly fell from 8.7 to 2.1 per 1,000 deliveries by counting fetal movements and taking prompt actions for further evaluation of fetal well-being when the pregnant women did not perceive 10 movements within a two hour time frame. The mean interval time for 10 perceived fetal movements was 20.9+/−18.1 minutes (mean +/−standard deviation). The number of antepartum testing (nonstress test, contraction stress test, biophysical profile) prompted by fetal movement count increased by 13%. Regardless of the counting methods used, this and other existing studies suggested an improvement in perinatal outcomes with early identification of decreased fetal activity. There are other on-going research activities to optimize the benefit of kick count, the information of which are readily available to those of ordinary skill and not discussed here for simplicity.

In general, the observation may be done at approximately the same time each day, preferably when the baby is usually very active or after mother has had a meal or snack. The mother may note the starting time and begin counting fetal movements (rolls, kicks, punches, turns), excluding hiccups, and continue counting until a certain number of movements are noted. Following the Moore and Piacquadio's study, perception of 10 distinct movements in a period of up to 2 hours may be considered reassuring. Once 10 movements have been perceived, the count may be discontinued.

Regardless of the type of protocol selected for a kick count, the efficacy of the kick count is affected by the accuracy in recording fetal movements during the kick count. Thus, there is a need for a device that records the time taken for a predetermined number of fetal movements, and also provides a bonding experience for the mother and her partner with the fetus during the pregnancy period. Traditionally, kick count chart was used where the information about the kick count session is recorded manually and, as a consequence, noncompliance has been a significant issue. Thus, there is also a need for a simple, user friendly device for registering and charting fetal movements, where the user may operate the device in one of the multi language settings. A pregnant woman may report the stored kick times to the prenatal care clinic or obstetrical care provider.

### OBJECTIVES OF THE INVENTION

It is an object of this invention to provide methods of kick count, in particular counting, timing and recording a predetermined number of fetal movements using the count-to-ten protocol, e.g., 10 fetal movements over 2 hours, as a means for fetal well-being monitoring.

It is another object of this invention to provide a simple method of fetal movement counting and charting that can be used at any time of the day, at any stage of pregnancy to enhance the compliance with the kick count protocol.

It is yet another object of this invention to incorporate voice and audio technology to simplify fetal movement counting.

It is a further object of this invention to provide incentive for a pregnant woman to perform fetal movement counting by incorporating automatic audio playback of the fetal heart sounds at the end of each counting session. The invention also incorporates days counter for the user to enter the pregnancy progress and update it at any time.

### SUMMARY OF THE INVENTION

The present invention provides methods and a device for charting fetal movements. The user of the device records each movement (or kick) by pressing a button on the device. The device records the time interval between the first kick and the tenth kick in each kick count session, where the elapsed time interval is referred to as “KickTime” hereinafter. The device stores ten KickTimes so that the user can review the frequency of the kicks from one sequence to the next. In addition, the device has a sound recording and playback function that allows the user to record the fetal heartbeat sound for playback. The device includes audible instructions to inform the user how to operate the device in each mode. The device also stores and updates the weeks and days of the woman's pregnancy progress.

In one aspect of the present invention, a device for charting fetal movements includes: an input member actuatable by a user to input fetal movement information into the device, the information corresponding to one or more perceived fetal movements; a kick counter for recording the fetal movement information during a kick count session, the kick count session being a period for recording a preset number of fetal movements; and a display screen for displaying icons during the kick count session, wherein each icon corresponds to one of the perceived fetal movements. The user of the device can count the perceived fetal movements recorded during the kick count session by counting the icons displayed during the kick count session.

In another aspect of the present invention, a method for charting fetal movements using a device that has a display screen includes steps of: causing the device to receive fetal movement information from the user thereof, the information corresponding to one or more perceived fetal movements; causing the device to record the fetal movement information during a kick count session, the kick count session being a period for recording a preset number of fetal movements; and causing the device to display icons on the display screen during the kick count session, wherein each icon corresponds to one of the perceived fetal movements. The user of the device can count the perceived fetal movements recorded during the kick count session by counting the icons displayed during the kick count session.

These and other features, aspects and advantages of the present invention will become better understood with reference to the following drawings, description and claims.

### BRIEF DESCRIPTION OF THE DRAWINGS:

FIGS. 1A-1C are a front, a side and a back elevation of a kick-count device, respectively, in accordance with the present invention.

FIG. 2A is an exemplary display on the device of FIG. 1A in a “kick” mode for recording the times of fetal movements.

FIG. 2B is an exemplary display on the device of FIG. 1A in a “play animation” mode for playing an animation upon completion of the kick mode.

FIG. 2C is an exemplary display on the device of FIG. 1A in a “kick memory” mode for reviewing KickTimes recorded in the kick mode.

FIG. 2D is an exemplary display on the device of FIG. 1A in a “play” mode for playing a fetal heartbeat sound recorded therein.

FIG. 2E is an exemplary display on the device of FIG. 1A in a “record” mode for recording a fetal heartbeat sound to be played in the play mode.

FIG. 2F is an exemplary display on the device of FIG. 1A in a “setup” mode for inputting information of the current stage of pregnancy, selecting a language and updating a “Days To Go” countdown.

FIG. 3 is a functional block diagram of the device depicted in FIG. 1A.

FIGS. 4A-4B show a flow chart illustrating an operational sequence of the modes depicted in connection with FIGS. 2A-2F.

FIGS. 5A-5C show a system flowchart of the device depicted in FIG. 1A.

FIG. 6 shows a flow chart illustrating the steps that may be carried out to count kicks using the device depicted in FIG. 1A according to the present invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Broadly, the present invention provides a device that is intended to help a pregnant woman chart fetal movements. The device may help the pregnant woman record and evaluate the elapsed time for ten perceived fetal movements to occur in a kick count session. The user of the device may record each movement (or kick) by pressing a button on the device and measure the KickTime of the kick count session. The device may store data of ten KickTimes so that the user can review the frequency of the kicks from one sequence to the next. In addition, the device may have a sound recording and playback function that enables the user to record and play a fetal heartbeat sound. The device may also store and update the weeks and days of the woman's pregnancy progress.

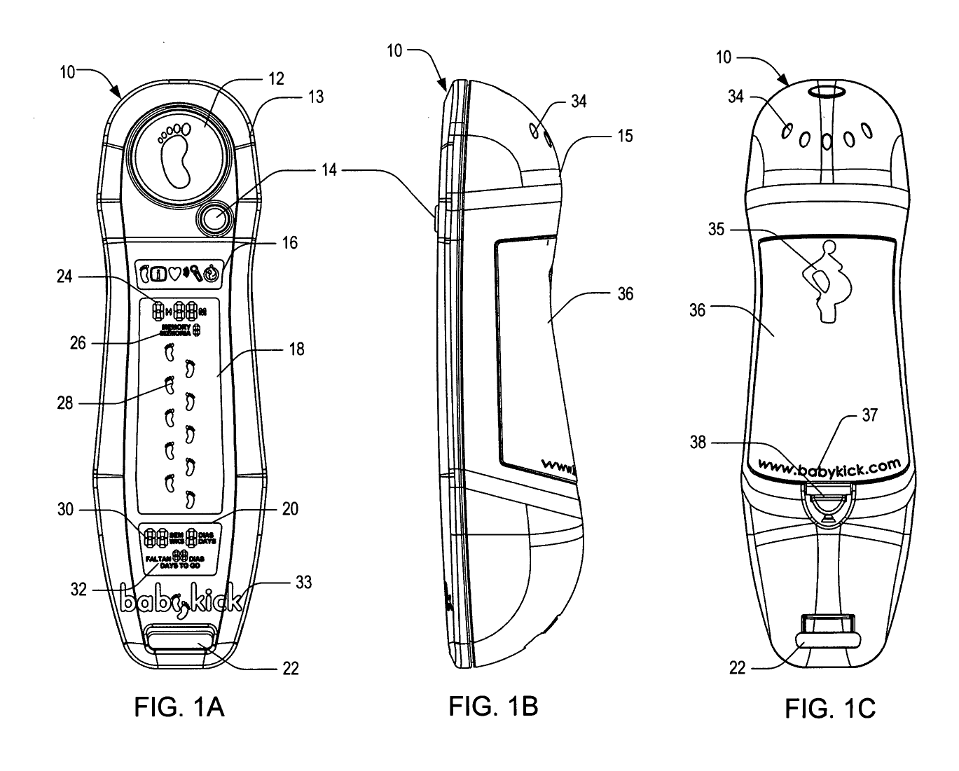

FIG. 1A is a front elevation of a kick-count device 10 in accordance with the present invention. As illustrated, the device 10 may include: an input member including an EVENT button 12 for recording events and a MODE button 14 for switching between modes; a mode window 16 for indicating the active mode; a kick count window 18 that includes a time display 24, a memory number display 26, and a 10-footprint display 28; a pregnancy progress window 20 that includes a week-and-day counter 30 and a day counter 32. (Hereinafter, the terms footprints and feet are used interchangeably.) The week-and-day counter 30 may indicate a count forward of the pregnancy in weeks and days from the inception of pregnancy, while the day counter 32 may indicate a countdown of ‘days-to-go’ during the last 99 days of the pregnancy. As will be explained later, the words “DAYS TO GO” of the day counter 32 may also be used to select the language by the user of the device 10. The device 10 may have one LCD screen that is masked by a front enclosure 13 to be perceived as three separate windows 16, 18 and 20. The LCD screen may be also used to display a digital image of the baby's ultrasound scan. Alternatively, a printout of the digital image may be slotted into a clear plastic pouch, where the pouch is attached to the front enclosure 13 or the back enclosure 15 (FIG. 1B).

The device 10 may also have a hole 22 through the enclosure to attach a strap for a key ring, wrist strap, necklace or the like. In an alternative embodiment, the device 10 may have a clip feature (not shown in FIG. 1A) so that it can be attached to a belt, clothing or a bag.

The website address 37 (shown in FIG. 1C), Voikex™ logo 35 (shown in FIG. 1C) and device name 33 may be located on the device 10. These graphics may be added as a part of the molding process or printed onto the device 10. The EVENT button 12 may be concave and level with the enclosure at the edges. The EVENT button 12 may be made of an elastomeric material, while the MODE button 14 may be made of a rigid plastic. In another alternative embodiment, the EVENT button 12 may be located at the top the device 10.

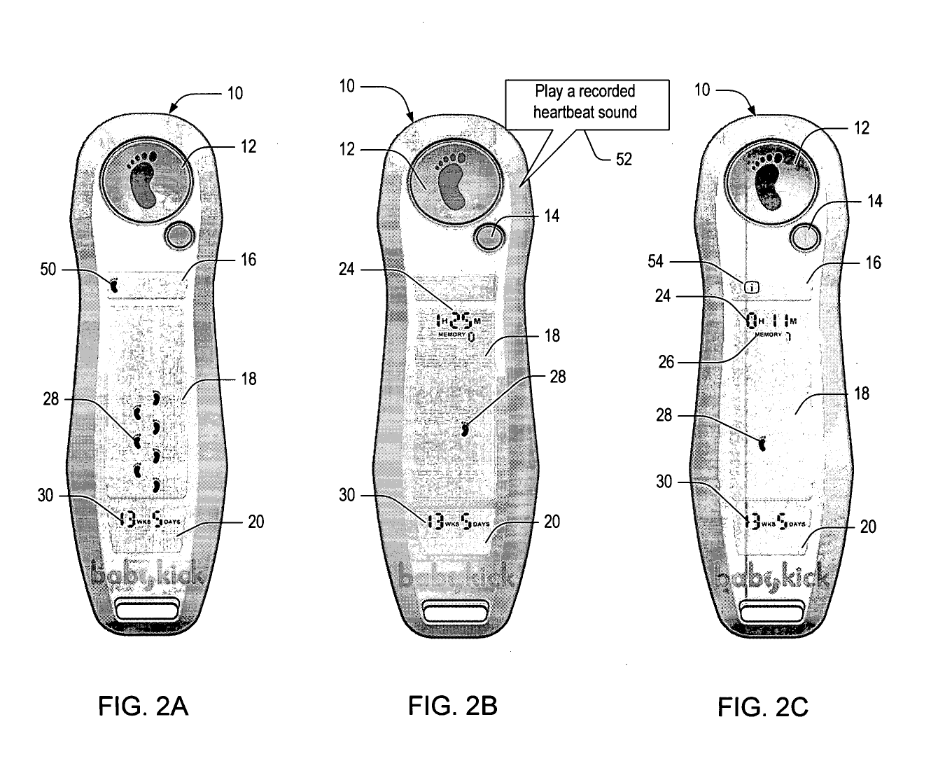

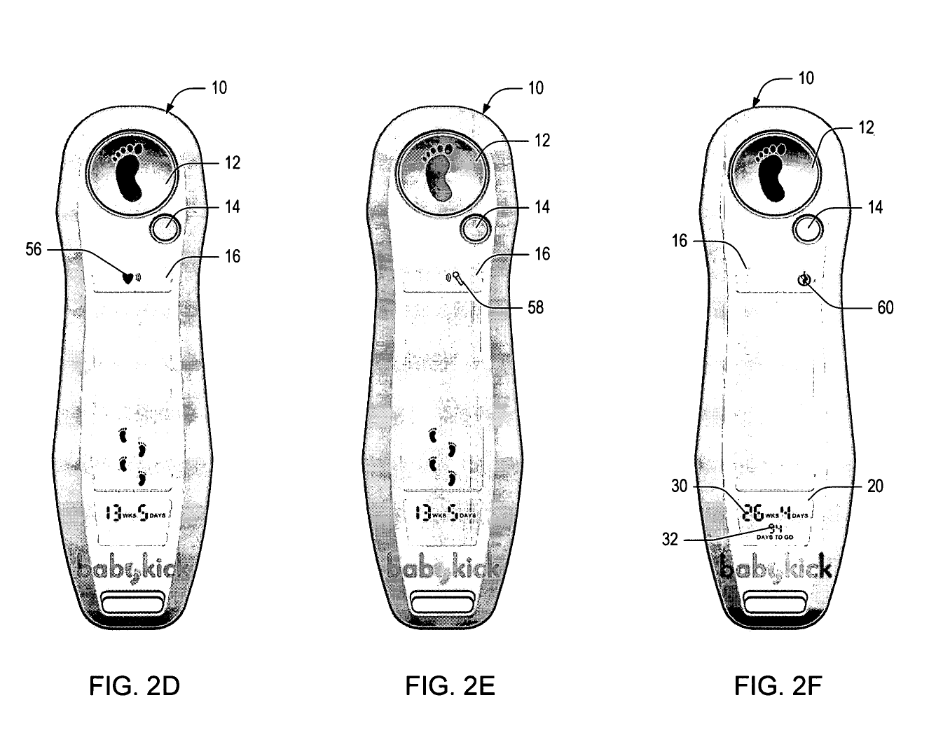

FIG. 1B is a side elevation of the kick-count device 10 depicted in FIG. 1A. As illustrated, the device 10 may include a back enclosure 15 that may be fixed to the front enclosure 13 securely and may not snap open when subjected to predictable impacts. The back enclosures 15 may include a removable battery cover 36 and holes 34 for a speaker located therewithin. The speaker, not shown in FIGS. 1A-1C, may be used to record a recognizable heartbeat sound from a Doppler heartbeat audio instrument and play the recorded sound. Further detail of the speaker will be given in connection with FIG. 2E. The front and back enclosure 13, 15 may have a pregnancy totem shape reminiscent of pregnant abdomen with a gentle female curvature.

FIG. 1C is a back elevation of the kick-count device 10 depicted in FIG. 1A. As illustrated, the back enclosure 15 may include: holes 34 for the speaker; a removable battery cover 36; and a battery cover opener 38 that opens the removable battery cover 36.

The device 10 may have the following (operational) modes; kick mode, play animation mode, kick memory mode, play mode, record mode and setup mode. The device may include audible instructions for each mode that will play after a predetermined time delay. These instructions may inform the user in the correct language how to operate the device in that particular mode. The user of the device 10 may press the MODE button 14 to switch from one mode to another while one of the icons in the mode window 16 may be highlighted to indicate the current mode. FIG. 2A is an exemplary display on the device 10 in a “kick” mode for recording the times of fetal movements. The device 10 may default to the kick mode when awaken from an idle state. As illustrated, a kick icon 50 may be highlighted in the mode window 16 to indicate that the device is currently operating in the kick mode. In this mode, the user of the device (preferably the mother) may record the times of fetal movements (or, shortly, kicks) by pressing the EVENT button 12 each time that the mother perceives a fetal movement. In the kick mode (and other modes described in FIGS. 2B-2F), the week-and-day counter 30 of the pregnancy progress window 20 may display a count forward from inception in weeks and days. If the current date is in the last 99 days of the pregnancy, the day counter 32 may display a count down to delivery in days.

In each (kick count) session, the user may record ten consecutive kicks. To indicate the number of kicks recorded in the current session, the kick count window 18 may display the images of footprint 28. When a kick is recorded by pressing the EVENT button 12, another image of the footprint 28 may be added to the kick count window 18. After 10 kicks have been recorded, the time taken to count these ten kicks (or equivalently, KickTime) may be displayed on the time display 24 of the kick window 18, as illustrated in FIG. 2B. FIG. 2B is an exemplary display on the device 10 in a “play animation” mode. Upon completion of the kick mode in FIG. 2A, the device 10 may default to the play animation mode, wherein the animated footprints 28 may walk up the kick window 18 along with a heartbeat sound for ten seconds as indicated in a text bubble 52. The fetal heartbeat sound may be recorded in the “record” mode as explained in connection with FIG. 2E. The play animation mode may alert the user that ten kicks have been recorded and one session is over.

As mentioned above, the user may record ten kicks in each session. The number “ten” has been selected to utilize the protocol recommended by American College of Obstetricians and Gynecologists (ACOG): 10 fetal movements (kicks) in 2 hours. However, it should be apparent to those of ordinary skill that the device 10 may be designed to record other suitable number of fetal movements in each session depending upon the kick count protocol.

As mentioned, upon completion of the kick mode (FIG. 2A), the device 10 may enter into the play animation mode (FIG. 2B) automatically. Likewise, once the animation is complete, the device 10 may default to a “kick memory” mode. FIG. 2C is an exemplary display on the device 10 in the kick memory mode for reviewing the KickTimes recorded in the kick mode in FIG. 2A. As illustrated in FIG. 2C, a memory icon 54 may be highlighted in the mode window 16 to indicate that the device is currently operating in the kick memory mode. In this mode, the device 10 may display ten KickTime records one at a time. The EVENT button 12 may be used to scroll through the ten KickTime records. For each KickTime record displayed, the corresponding memory number (or equivalently, a stack number of the displayed KickTime record in the ten KickTime memory) may be displayed on the memory number display 26. To help the user trace the memory number, a footprint 28 may be highlighted on the kick count window 18. The user may press the MODE button 14 to exit the kick memory mode and switch to a “play” mode.

FIG.2D is an exemplary display on the device 10 in a play mode for playing a heartbeat sound recorded in the device 10. In this mode, the user may be able to play the fetal heartbeat sound, which is preferably a fetal heartbeat sound recorded from a Doppler instrument, by pressing down the EVENT button 12 for more than 0.15 seconds. As depicted in FIG. 2D, a play icon 56 may be used to indicate the device is currently operating in the play mode. The play icon 56 may flash during the playback of the sound recording or stay being highlighted if the device 10 does not have any recorded sound. The speaker volume may be controlled by pressing down the EVENT button 12 for more than 2 seconds during which the volume may toggle between 25% and 100% of the maximum level. Then, the user may set the volume to a desired level by releasing the EVENT button 12.

The user may press the MODE button 14 to exit the play mode and switch to a “record” mode. FIG. 2E shows an exemplary display on the device 10 in the record mode for recording the heartbeat sound to be played in the play mode. As illustrated, a record icon 58 may be displayed on the mode window 16 to indicate that the device 10 is currently operating in the record mode. The user may bring the device 10 to an obstetrical provider and record the fetal heartbeat sound from a Doppler instrument by pressing and holding the EVENT button 12. Initially, the device 10 may be programmed with a pre-recorded heartbeat sound. This may be erased when the user makes her first recording. Also, a new recording may automatically overwrite the previous one. The device 10 may record the heartbeat sound for 10 seconds. The record icon 58 may flash for 10 seconds from the start of recording and subsequently stay highlighted to indicate that the recording has completed. It should be apparent to those of skill in the art that the device 10 may be easily programmed to record more or less than 10 seconds.

The user may press the MODE button 14 to exit the record mode and switch to a “setup” mode. FIGS. 2F shows an exemplary display on the device 10 in the setup mode for inputting information of the current stage of pregnancy into the device 10. The setup mode has three submodes: “SetWeeks”, “SetDates” and “SetLanguage” submodes. When the device 10 enters into the SetWeeks submode, the number representing the “Weeks Pregnant” in the week-and-day counter 30 may flash indicating that the device 10 is operating in the SetWeeks submode. The user may press the EVENT button 12 to set the weeks, where the weeks may loop from 0 to 42 and back to 0. There may be a time delay before the device 10 switches from the SetWeeks submode to the SetDays submode. In the SetDays submode, the number representing the “Days Pregnant” (in addition to the weeks pregnant) in the week-and-day counter 30 may flash and the user may press the EVENT button 12 to set the days, wherein the days may loop from 0 to 6.

The device 10 maybe set to count forward from the inception of pregnancy as well as to count down until the completion of 40 weeks of pregnancy. If the user's input for the week-and-day counter 30 indicates that the current date is in the last 99 days of the pregnancy, the device 10 may additionally display the count-down days on the day counter 32 as illustrated in FIG. 2F.

There may be a time delay before the device 10 automatically switches from the SetDays submode to the SetLanguage submode. The words “DAYS TO GO” or “FALTAN _DIAS” of the day counter 32 may blink to indicate the currently selected language. The user may toggle between the languages by pressing the EVENT button 12 and select one by pressing the MODE button 14. Once a language is selected, the graphics on the windows 16, 18 and 20 may be displayed in the selected language.

When a new battery(ies) are installed in the device 10, the device may enter into the SetLanguage submode prompting the user to select a language. The user may exit the setup mode by pressing the MODE button 14. It should be apparent to those of ordinary skill that the device 10 may be designed to display other languages than English and Spanish. Also, the ordinary skill in the art would appreciate that the design engineer of the device 10 can easily change the shapes of the icons displayed on the display windows 16 and 18.

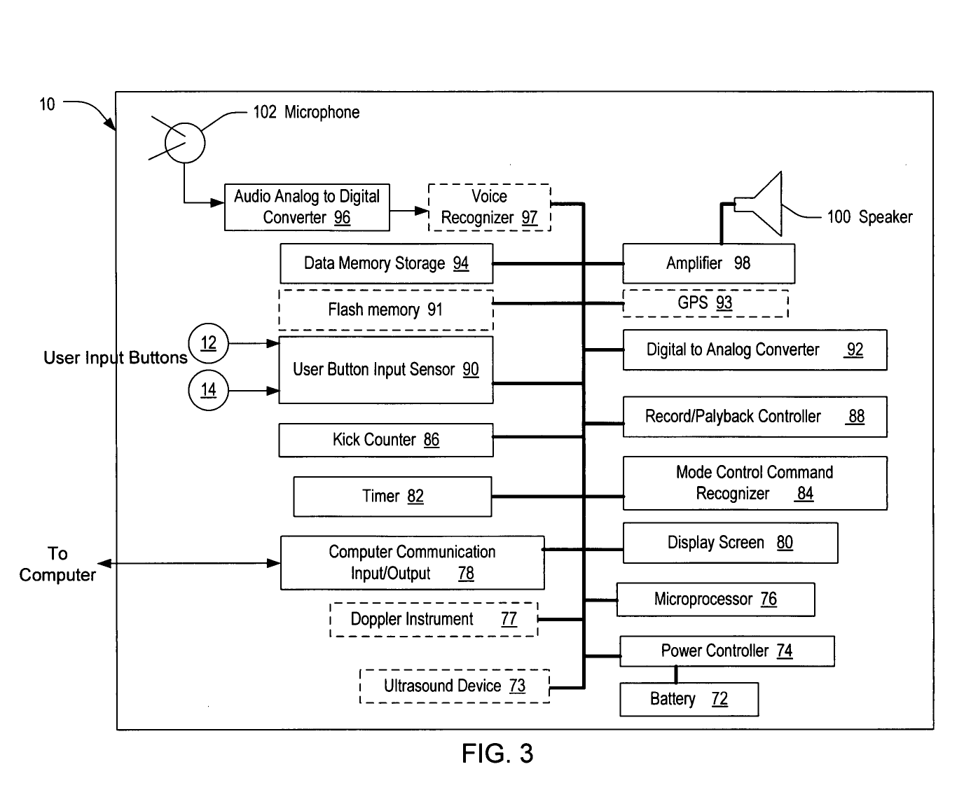

FIG. 3 is a functional block diagram of the device 10. The device 10 may include: a microphone 102 for receiving the fetal heartbeat sound from a Doppler instrument in the record mode; an analogue-to-digital converter 96 for converting the sound input received from the microphone 102 into a digital signal; a data memory storage (or, shortly, data storage) 94 for storing the converted digital signal; a digital-to-analogue converter 92 for converting the stored digital signal into an analogue signal; an amplifier 98 for amplifying the analogue signal received from the digital-to-analogue converter 92; a speaker 100 for playing the amplified analogue signal received from the amplifier 98; two buttons (EVENT and MODE buttons) 12, 14; a user button input sensor 90 for receiving the user's input through the two buttons 12, 14; a kick counter 86 for recording the input from the user in the kick mode (FIG. 2A); a timer 82 for timing events, such as kicks, and measuring the KickTime of a session; a record/playback controller 88 for controlling the length of a recorded sound and adjusting the volume-of the speaker 100; a mode control command recognizer 84 for receiving signals from the user button input sensor 90 and determining the current operational mode; a display screen 80, preferably a LCD screen, masked by the front enclosure 13 to appear as three separate windows 16, 18 and 20; a power controller 74 for receiving power from a battery(ies) 72 and switching to sleep mode when the device 10 is in the idle state; and a microprocessor 76 for controlling and orchestrating the elements of the device 10 shown in FIG. 3. (Hereinafter, the term “audio member” may collectively refer to the speaker 100 and the microphone 102.) It is noted that the speaker 100 may be also used as the microphone 102 and record other suitable sounds, such as a baby's first cry or words, as well as the fetal heartbeat sound. In an alternative embodiment, a solar panel may be used in place of the battery 72.

The data (memory) storage 94 may also store other information including names, obstetrical data such as fetal development milestones, personal medical and emergency information, medical visits, gestational age specific recommendation, organizer, calendar, chronometer and pediatric data, such as pediatric growth chart.

Optionally, the device 10 may include one or more of the followings: a computer communication Input/Output 78 for exchanging information, such as the sound and kick history recorded in the device 10, with a computer; a voice recognizer 97 for recognizing the user's voice to understand verbal commands and activating the device 10 in accordance with the commands; a built-in Doppler instrument 77 for picking up fetal heartbeat sound and playing/recording the sound; a built-in ultrasound device 73 for sensing and/or imaging fetal movement and, thereby, counting the fetal movements without or in addition/conjunction with the user's input via the EVENT button 12; a flash memory 91 for storing verbal instructions for use of the device 10; and a global positioning system (GPS) 93 for determining the user's current location and providing a navigational guidance to a destination, such as a hospital, for the user. In an alternative embodiment, the flash memory 91 may be included in the data memory storage 94.

It will be appreciated by those of the ordinary skill that elements illustrated in FIG. 3 may be modified in a variety of ways without departing from the spirit and scope of the present invention. For example, the mode control command recognizer 84 may be a firmware or software stored in the data memory storage 94 and executed by the microprocessor 76. The kick counter 86 may be implemented and executed in the similar way. Alternatively, the kick counter 86 may be combined with the mode control command recognizer 84 prior to storage in the data memory storage 94. In another example, the computer communication Input/Output 78 may be configured to communicate with various electronic devices, such as PDA or other suitable hand-held devices, as well as a computer.

The device 10 may use a conventional wireless/blue tooth technology to connect to a computer to upload or download the data stored in storage 94. The device 10 may also incorporate an MP3, writing instrument, compact mirror, watch, clock, pendant, bracelet, key chain and software for PDA or PC.

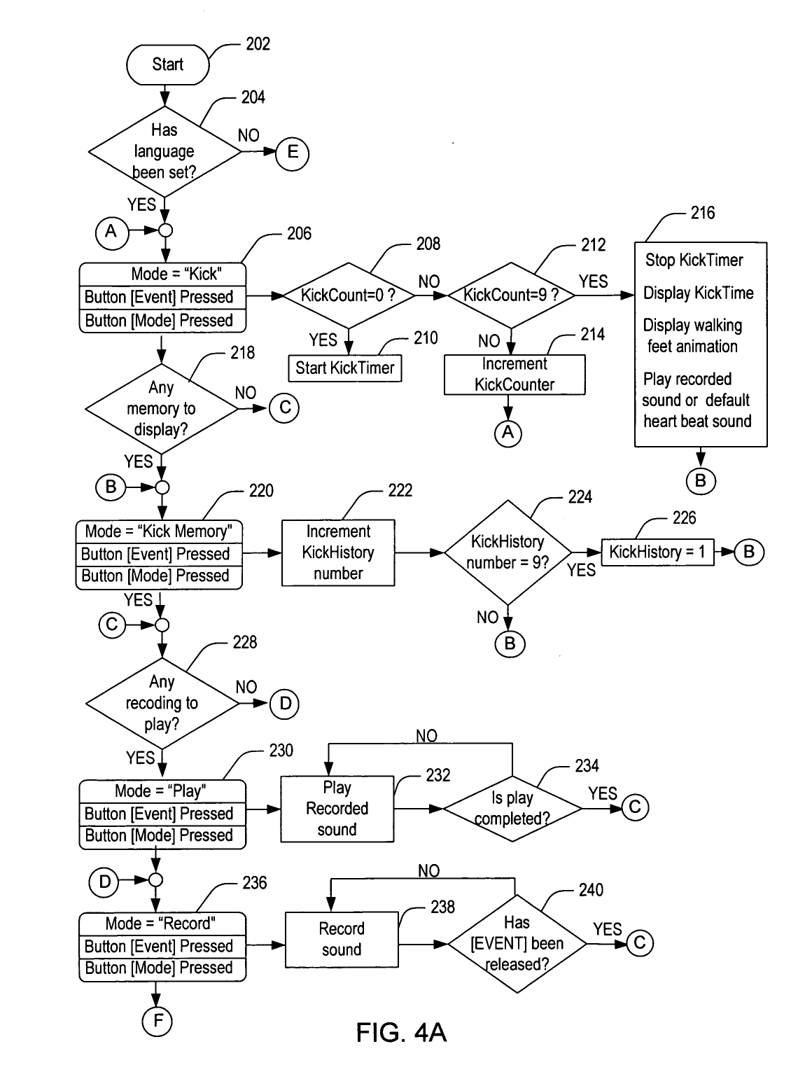

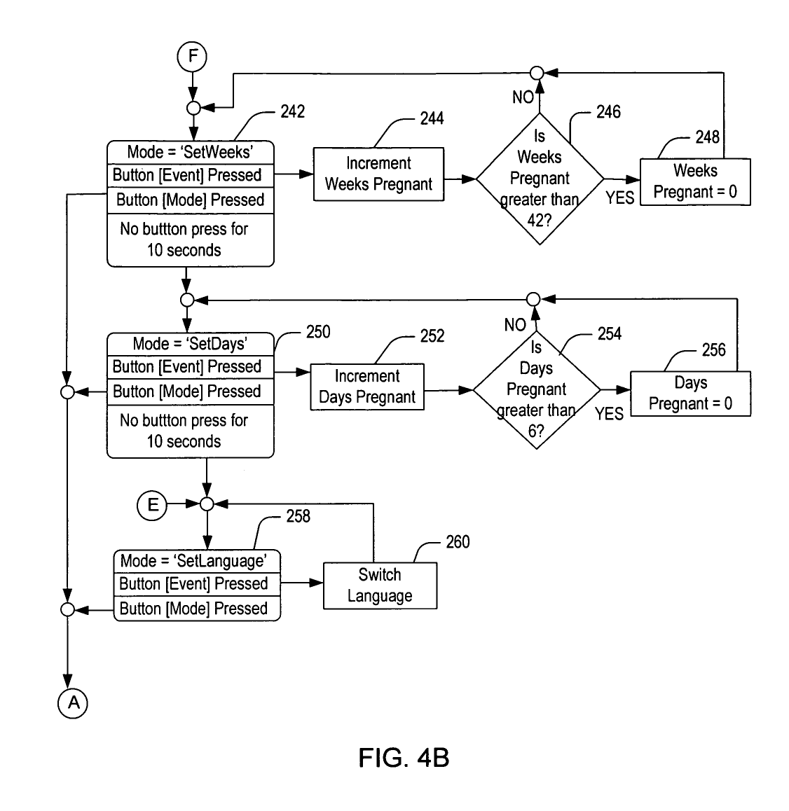

FIGS. 4A-4B show a flow chart illustrating the operational sequence of the modes depicted in connection with FIGS. 2A-2F. The process may start in a state 202. Next, in a state 204, the device 10 may check if the user has selected a language. Upon negative answer to the state 204, the process may advance to a state 258 (shown and illustrated in connection with FIG. 4B). Otherwise, the user may press the MODE button 14 to proceed to a state 206.

The state 206 may correspond to the kick mode. If the user presses the EVENT button 12 to record a kick, the kick counter 86 (FIG. 3) may determine if the kick is the first one of the current session. Upon positive answer to the state 208, the kick counter 86 may start the Timer 82 in a state 210, where the Timer 82 may operate to measure the KickTime of the current session. If the answer to the state 208 is negative, the process may advance to a state 212.

In the state 212, the kick counter 86 may determine if the current kick is the 10^{th} kick of the current session. Upon positive answer to the state 212, the device 10 may stop the Timer 82 and play the animation as described in connection with FIG. 2B. The animation may include displaying the KickTime on the time display 24, displaying a walking footprint animation 28 and playing a recorded heartbeat sound 52. Then, the process may proceed to a state 220. Upon negative answer to the state 212, the kick counter 86 may increment the kick count by one. Subsequently, the process may proceed to the state 206 to cause the device 10 to stay in the kick mode.

If the user presses the MODE button 14 in the state 206, the process may advance to a state 218. In the state 218, the device 10 may determine whether the user has logged ten KickTime records to be displayed, i.e., the user has completed ten sessions. If the device 10 has ten KickTime records stored in the data memory storage 94 (FIG. 3), the process may advance to a state 220. Otherwise the process may proceed to a state 228.

The state 220 may correspond to the kick memory mode. As described in connection with FIG. 2C, the user may press the EVENT button 12 to display the ten KickTime records one at a time. The EVENT button 12 may be used to scroll through the ten records. For each record displayed, a corresponding footprint 28 may be displayed on the kick count window 18. The record number for the corresponding KickTime record may be displayed on the record number display 26. Also, the time display 24 may be used to indicate the KickTime. Each time the user presses the EVENT button 12, the kickhistory number (or equivalently, record number) may be increased by one in a state 222. Then the process may proceed to a state 224.

In the state 224, the device 10 may determine if the current kickhistory number equals nine, i.e., the currently displayed KickTime record is the tenth one. Upon negative answer to the state 224, the process may proceed to the state 220. Otherwise, the kick counter 86 may reset the kickhistory number to 1. Subsequently, the process may advance to the state 220.

In the state 220, the user may press the MODE button 14 causing the process to advance to a state 228. In the state 228, the decision is made as to whether the data memory storage 94 (FIG. 3) has any sound recording to be played. Upon negative answer to the state 228, the process may proceed to a state 236. Otherwise, the process may proceed to a state 230.

The state 230 may correspond to the play mode. In the state 230, the user may press the EVENT button 12, causing the process to proceed to a state 232. In the state 232, the record/playback controller 88 (FIG. 3) may play the recorded fetal heartbeat sound. Next, in a state 234, the record/playback controller 88 may determine whether the play has completed. Upon positive answer to the state 234, the process may advance to the state 236. If the answer to the state 234 is NO, the process may proceed to the state 232.

The state 236 may correspond to the record mode. In the state 236, the user may press the EVENT button 12, causing the process to proceed to a state 238. In the state 238, the record/playback controller 88 (FIG. 3) may record the fetal heartbeat sound from a Doppler instrument by pressing and holding the EVENT button 12. Next, in a state 240, the record/playback controller 88 may determine whether the EVENT button 12 has been released. Upon positive answer to the state 240, the process may advance to the state 228. If the answer to the state 240 is NO, the process may proceed to the state 238.

The user may press the MODE button 14 in the state 236, causing the process to proceed to a state 242 in FIG. 4B. The state 242 may correspond to the SetWeeks submode. In the state 242, the user may press the EVENT button 12 causing the process to proceed to a state 244. In the state 244, the device 10 may increase the Weeks Pregnant in the week-and-day counter 30 by one each time the user presses the EVENT button 12. Next, in a state 246, a determination may be made as to whether the Weeks Pregnant is greater than forty two. Upon negative answer to the state 246, the process may proceed to the state 242. Otherwise, the process may advance to a state 248. In the state 248, the Weeks Pregnant may be set to zero. Then, the process may proceed to the state 242. The user may press the MODE button in the state 242, causing the process to proceed to the state 206.

If the user does not press any button for a preset time interval, preferably for 10 seconds, in the state 242, the process may proceed to a state 250. The state 250 may correspond to the SetDays submode. In the state 250, the user may press the EVENT button 12 causing the process to proceed to a state 252. In the state 252, the device 10 may increase the Days Pregnant in the week-and-day counter 30 by one each time the user presses the EVENT button 12. Next, in a state 254, a determination may be made as to whether the DueDays is greater than six. Upon negative answer to the state 254, the process may proceed to the state 250. Otherwise, the process may advance to a state 256. In the state 256, the Days Pregnant may be set to zero. Then, the process may proceed to the state 250. The user may press the MODE button 14 in the state 250, causing the process to proceed to the state 206.

If the user does not press any button for a preset time interval, preferably for 10 seconds, in the state 250, the process may proceed to a state 258. The state 258 may correspond to the SetLanguage submode. In the state 258, the user may press the EVENT button 12 causing the process to proceed to a state 260. In the state 260, the user may select a language as described in connection with FIG. 2F. Then, the process may advance to the state 258. The user may press the MODE button 14 in the state 258, causing the process to proceed to the state 206.

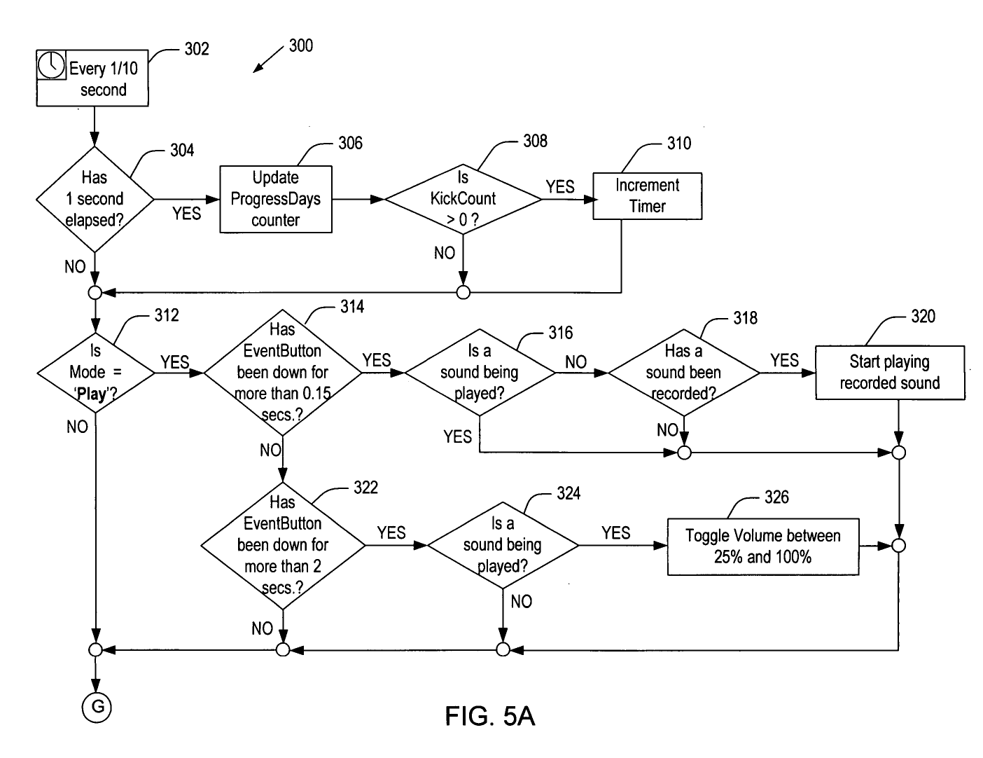

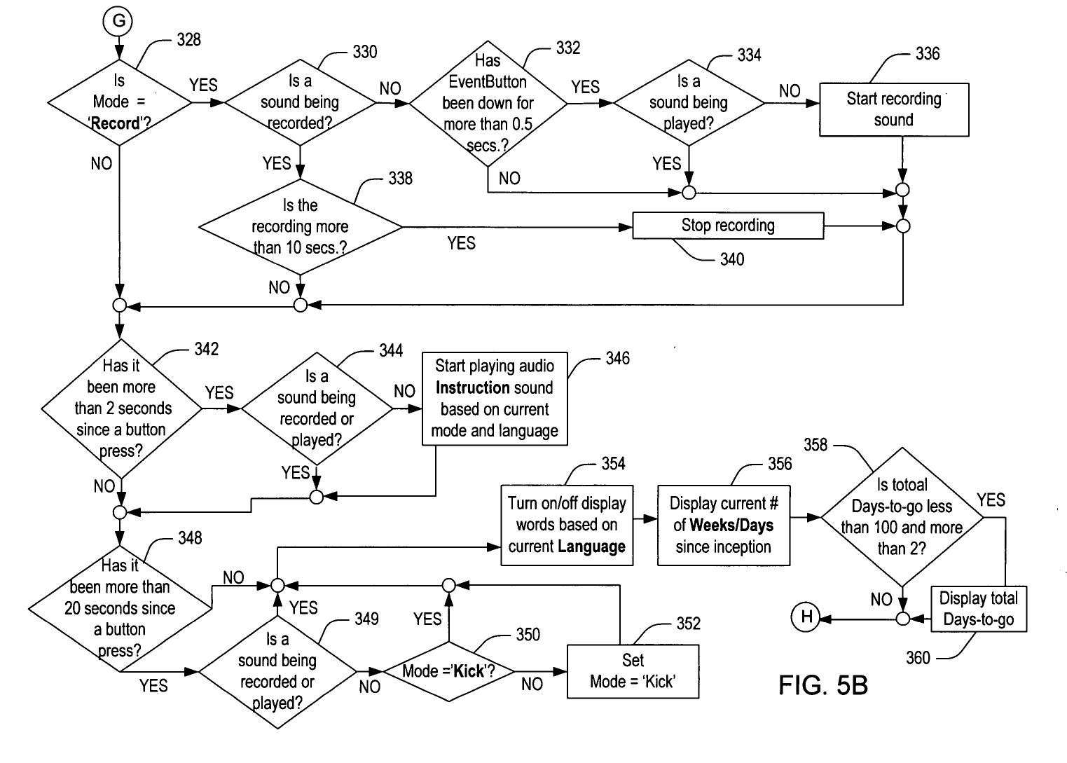

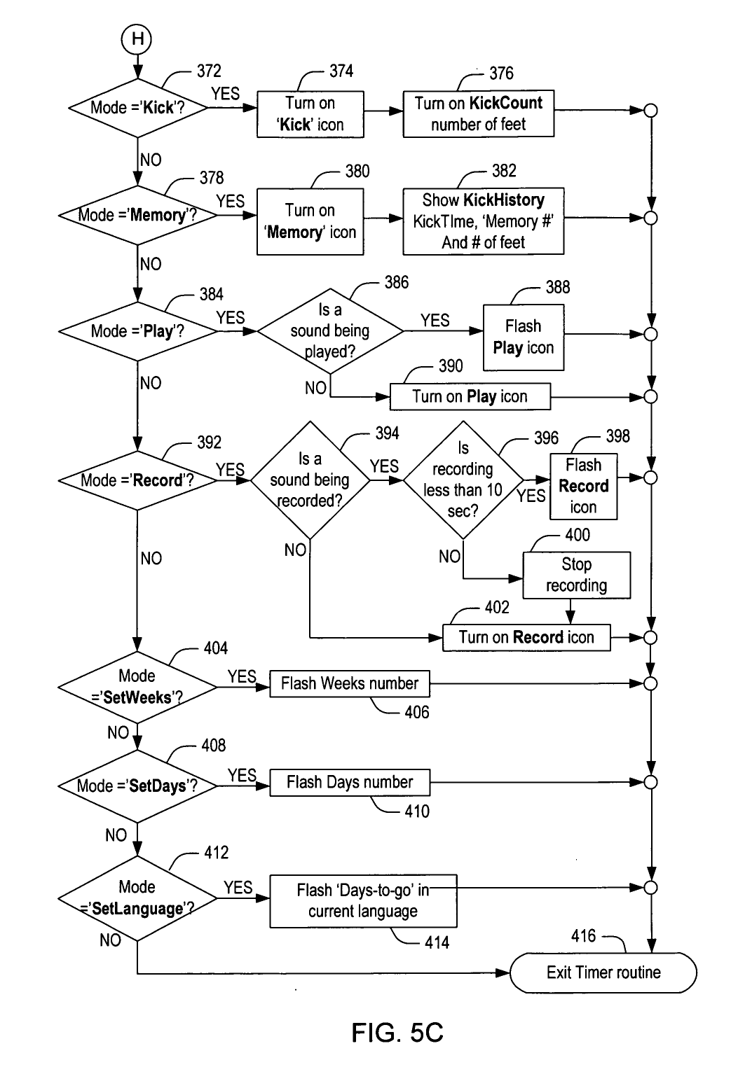

FIGS. 5A-5C show a system flowchart shown at 300 of the device depicted in FIG. 1A. The process may start at every 1/10 second in a state 302, where the timer 82 (FIG. 3) may provide the clock time. Next, the process may advance to a state 304, where a determination may be made as to whether one second has elapsed. Upon positive answer to the state 304, the progress may advance to a state 306. In the state 306, the device 10 may update the ProgressDays counter, where the ProgressDays collectively refer to the “Weeks and Days Pregnant” of the week-and-day counter 30 and the count-down of days-to-go of the day counter 32. Next, in a state 308, another determination is made as to whether the KickCount is greater than zero, i.e., the user has started recording the kicks in a session. Upon positive answer to the state 308, the Timer may be incremented in a state 310. Then, the process may advance to a state 312. If the answer to the state 308 is negative or the answer to the state 304 is negative, the process may proceed to the state 312.

In the state 312, a decision may be made as to whether the current mode is the play mode. Upon positive answer to the state 312, the process may proceed to a state 314. In the state 314, the user button input sensor 90 (FIG. 3) may determine if the user has pressed down the EVENT button 12 for more than 0.15 seconds. If the answer to the state 314 is YES, the process may proceed to a state 316. In the state 316, a determination may be made as to whether the data memory storage 94 contains a heartbeat sound to be played. If the storage 94 contains a sound, the record/playback controller 88 may play the sound through the speaker 100 in a state 320. Then, the process may proceed to a state 328 (FIG. 5B). If the answer to the state 316 is positive or the answer to the state 318 is negative, the process may advance to the state 328.

Upon negative answer to the state 314, the process may proceed to a state 322. In the state 322, the user button input sensor 90 may determine if the user has pressed down the EVENT button 12 for more than 2 seconds. If the answer to the state 322 is YES, the process may proceed to a state 324. In the state 324, a determination may be made as to whether the data memory storage 94 contains a heartbeat sound to be played. If the storage 94 contains a sound, the speaker volume may toggle between 25% and 100% of the maximum level in a state 326. Then, the process may proceed to the state 328 in FIG. 5B. Upon negative answer to the state 322, the process may also proceed to the state 328.

In the state 328, a decision may be made as to whether the current mode is the record mode. Upon positive answer to the state 328, the process may proceed to a state 330. In the state 330, a determination may be made as to whether a sound is being recorded. If the answer to the state 330 is NO, the process may proceed to a state 332. In the state 332, the mode control command recognizer 84 may determine if the user has pressed down the EVENT button 12 for more than 0.5 seconds. If the user has pressed down the EVENT button 12 more than 0.5 seconds, the process may proceed to a state 334 to determine whether a heartbeat sound is being played. Upon negative answer to the state 334, the process may proceed to a state 336 to start recording a sound. Then, the process may proceed to a state 342. If the answer to the state 332 is negative or the answer to the state 334 is positive, the process may also proceed to the state 342.

Upon positive answer to the state 330, the process may proceed to a state 338. In the state 338, a determination may be made as to whether the recording is more that 10 seconds. If the answer to the state 338 is YES, the process may proceed to a state 340 to stop recording. Subsequently, the process may proceed to the state 342. If the answer to the state 328 is negative, the process may also proceed to the state 342.

In the state 342, the user button input sensor 90 (FIG. 3) may determine if more than 2 seconds has elapsed since a button has been pressed. If the answer to the state 342 is YES, the process may proceed to a state 344. In the state 344, a determination may be made as to whether a sound is being recorded or played. If the answer to the state 344 is NO, the process may proceed to a state 346. In the state 346, the record/playback controller 88 may start playing audio instruction sound based on the current mode and language. Then, the process may proceed to a state 348. If the answer to the state 342 is NO or the answer to the state 344 is YES, the process may also proceed to the state 348.

In the state 348, the user button input sensor 90 (FIG. 3) may determine if more than 20 seconds has elapsed since a button has been pressed. If the answer to the state 348 is YES, the process may proceed to a state 349. In the state 349, a determination is made whether a recording is being played. If the answer to the state 349 is NO, the process may proceed to a state 350. In the state 350, a determination is made whether the current mode is the kick mode. If the answer to the state 350 is NO, the process may proceed to a state 352. In the state 352, the current mode is set to the kick mode. Then, the process may advance to a state 354. If the answer to the state 348 is NO or the answer to the state 349 is YES or the answer to the state 350 is YES, the process may proceed to a state 354.

In the state 354, the display screen 80 may turn on/off the displayed words based on the current language. Next, in a state 356, numbers of weeks and days may be displayed on the week-and-day counter 30. Then, the process may advance to a state 358. In the state 358, a determination is made whether the countdown days-to-go is less than 100 and more than 2. If the answer to the state 358 is YES, the process may proceed to the state 360 to display the countdown days-to-go on the day counter 32. Then, the process may proceed to a state 372 (FIG. 5C). If the answer to the state 358 is NO, the process may also proceed to the state 372.

In the state 372, a determination is made whether the current mode is the kick mode. If the answer to the state 372 is YES, the kick icon 50 (FIG. 2A) may be turned on (or equivalently, highlighted) in a state 374. Subsequently, in a state 376, footprints 28 may be displayed on the kick count window 18. Then, the process may proceed to a state 416. If the answer to the state 372 is NO, the process may proceed to a state 378.

In the state 378, a determination is made whether the current mode is the kick memory mode. If the answer to the state 378 is YES, the memory icon 54 (FIG. 2C) may be turned on in a state 380. Subsequently, in a state 382, the kickhistory for each KickTime record may be displayed, where the kickhistory may include a KickTime, a record number and a footprint corresponding to the record number. Then, the process may proceed to a state 416. If the answer to the state 378 is NO, the process may proceed to a state 384.

In the state 384, a determination is made whether the current mode is the play mode. If the answer to the state 384 is YES, a determination may be made as to whether a sound is being played in a state 386. If the answer to the state 386 is YES, the play icon 56 (FIG. 2D) may flash in a state 388. Subsequently, the process may proceed to the state 416. If the answer to the state 386 is NO, the play icon 56 may be highlighted in a state 390. Then, the process may proceed to the state 416. If the answer to the state 384 is NO, the process may proceed to a state 392.

In the state 392, a determination is made whether the current mode is the record mode. If the answer to the state 392 is YES, a determination may be made as to whether a sound is being recorded in a state 394. If the answer to the state 394 is YES, a determination may be made as to whether the recording is less than 10 seconds in a state 396. Upon positive answer to the state 396, the process may proceed to a state 398. In the state 398, the record icon 58 may flash. Then, the process may proceed to the state 416. Upon negative answer to the state 396, the record/playback controller 88 may stop recording in a state 400. Then, the process may proceed to the state 416. If the answer to the state 394 is NO, the record icon 58 may be highlighted. Subsequently, the process may proceed to the state 416. If the answer to the state 392 is NO, the process may proceed to a state 404.

In the state 404, a determination is made whether the current mode is the SetWeeks submode. If the answer to the state 404 is YES, the Weeks Pregnant of the week-and-day counter 30 flash in a state 406. Then, the process may proceed to the state 416. If the answer to the state 404 is NO, the process may proceed to a state 408.

In the state 408, a determination is made whether the current mode is the SetDays submode. If the answer to the state 408 is YES, the DueDays of the week-and-day counter 30 may flash in a state 410. Then, the process may proceed to the state 416. If the answer to the state 408 is NO, the process may proceed to a state 412.

In the state 412, a determination is made whether the current mode is the SetLanguage submode. If the answer to the state 414 is YES, the word “days to go” of the day counter 30 may flash in the current language in a state 414. Then, the process may proceed to the state 416. If the answer to the state 412 is NO, the process may proceed to the state 416. In the state 416, the process for every 0.1 second started in the state 302 in FIG. 5A may exit.

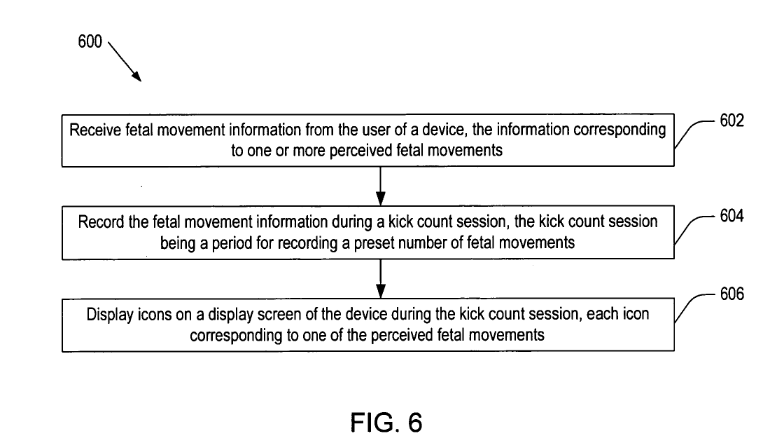

FIG. 6 is a flow chart shown at 600 illustrating the steps that may be carried out to count kicks using the device 10 according to the present invention. In a state 602, the user of the device 10 may press the EVENT button 12 each time a fetal movement is perceived in a kick count session. Then, the device 10 may record the fetal movements up to a preset number, preferably ten, in the kick count session. During the kick count session, the device 10 may display icons on its display screen, where one additional icon is displayed each time the user presses the EVENT button 12 in a state 606. As a consequence, the user can count the perceived fetal movements recorded in the kick count session by counting the displayed icons.

It should be understood, of course, that the foregoing relates to exemplary embodiments of the invention and that modifications may be made without departing from the spirit and scope of the invention as set forth in the following claims.

1. A device for charting fetal movements perceived by a pregnant woman comprising:
an input member actuatable by a user to input fetal movement information into said device, the information corresponding to one or more perceived fetal movements and to be input by actuating said input member each time a fetal movement is perceived by a pregnant woman;
a kick counter for recording the fetal movement information during a kick count session, the kick count session being a period for recording a preset number of fetal movements; and
a display screen for displaying icons during the kick count session, each icon corresponding to one of the perceived fetal movements,
whereby the user is able to count the perceived fetal movements recorded during the kick count session by counting the icons displayed during the kick count session.

2. A device for charting fetal movements as recited in claim 1, further comprising:
a timer for measuring the duration of the period, KickTime; and
a data storage for storing the KickTime;
wherein the user actuates the input member to cause said display screen to display the KickTime stored in said data storage.

3. A device for charting fetal movements as recited in claim 2, further comprising:
a Doppler instrument for picking up a fetal heartbeat sound and storing the heartbeat sound into said data storage.

4. A device for charting fetal movements as recited in claim 2, further comprising an audio member for receiving a sound and communicating the sound to said data storage, said audio member operable as a speaker.

5. A device for charting fetal movements as recited in claim 4, wherein the sound is one selected from the group consisting of heartbeat sound from a Doppler instrument, baby's first cry and baby's first words.

6. A device for charting fetal movements as recited in claim 4, wherein each of the icons is in the form of a footprint and wherein, upon completion of the kick count session, said display screen sequentially displays the icons to perform an animation of feet walking and said audio member operates as a speaker to play the sound during the animation.

7. A device for charting fetal movements as recited in claim 4, further comprising a voice recognizer coupled to said audio member and operable to recognize the user's voice command.

8. A device for charting fetal movements as recited in claim 1, further comprising a global positioning system for determining the user's current location and providing a navigational guidance for the user.

9. A device for charting fetal movements as recited in claim 1, further comprising a processor, wherein the information further includes pregnancy progress data in weeks and days, wherein said processor calculates a countdown of days-to-go using the pregnancy progress data, and wherein said display screen displays the countdown if the countdown is less than a predetermined number.

10. A device for charting fetal movements as recited in claim 1, wherein said display screen has multiple language settings.

11. A device for charting fetal movements as recited in claim 1, wherein said display screen displays a digital image of a baby's ultrasound scan.

12. A device for charting fetal movements as recited in claim 1, further comprising an Input/Output for communicating with an electronic device.

13. A device for charting fetal movements as recited in claim 12, wherein the electronic device is one selected from the group consisting of a computer and a personal digital assistant (PDA).

14. A device for charting fetal movements as recited in claim 12, wherein the Input/Output is a wireless device.

15. A device for charting fetal movements as recited in claim 1, further comprising a flash memory for storing one or more audio instructions for use of the device.

16. The device for charting fetal movements as recited in claim 1, further comprising:
an ultrasound device for sensing and imaging the perceived fetal movements.

17. A method for charting fetal movements perceived by a pregnant woman using a device that includes a display screen and an input member, comprising:
causing the device to receive fetal movement information from the user thereof, the information corresponding to one or more perceived fetal movements and to be input by actuating said input member each time a fetal movement is perceived by a pregnant woman;
causing the device to record the fetal movement information during a kick count session, the kick count session being a period for recording a preset number of fetal movements; and
causing the device to display icons on the display screen during the kick count session, each icon corresponding to one of the perceived fetal movements,
whereby the user is able to count the perceived fetal movements recorded during the kick count session by counting the icons displayed during the kick count session.

18. A method for charting fetal movements as recited in claim 17, further comprising:
causing the device to measure the duration of the period, KickTime; and
causing the device to store the KickTime in a data storage thereof; and
causing the device to display the stored KickTime on the display screen.

19. A method for charting fetal movements as recited in claim 17, further comprising:
recording a heartbeat sound into a data storage of the device; and
causing the device to play the heartbeat sound.

20. A method for charting fetal movements as recited in claim 19, wherein each of the icons is in the form of a footprint, further comprising:
upon completion of the kick count session, causing the device to display sequentially the icons on the display screen to perform an animation of feet walking; and
causing the device to play the heartbeat sound during the animation.

21. A method for charting fetal movements as recited in claim 17, wherein said device further includes a voice recognizer, further comprising:
causing the voice recognizer to recognize the user's voice command.

22. A method for charting fetal movements as recited in claim 17, further comprising:
providing one or more verbal instructions for use of the device.

23. A method for charting fetal movements as recited in claim 17, wherein said device further includes an ultrasound device, further comprising, prior to the step of causing the device to record:
causing the ultrasound device to sense and image the perceived fetal movements.

24. A device for charting fetal movements, comprising:
an input member actuatable by a user to input fetal movement information into said device, the information corresponding to one or more perceived fetal movements;
a kick counter for recording the fetal movement information during a kick count session, the kick count session being a period for recording a preset number of fetal movements;
a display screen for displaying icons during the kick count session, each icon corresponding to one of the perceived fetal movements,
a timer for measuring the duration of the period, KickTime; and
a data storage for storing the KickTime;
an audio member for receiving a sound and communicating the sound to said data storage, said audio member operable as a speaker,
wherein the user actuates the input member to cause said display screen to display the KickTime stored in said data storage and wherein each of the icons is in the form of a footprint and wherein, upon completion of the kick count session, said display screen sequentially displays the icons to perform an animation of feet walking and said audio member operates as a speaker to play the sound during the animation,
whereby the user is able to count the perceived fetal movements recorded during the kick count session by counting the icons displayed during the kick count session.

25. A method for charting fetal movements using a device that includes a display screen, comprising:
causing the device to receive fetal movement information from the user thereof, the information corresponding to one or more perceived fetal movements;
causing the device to record the fetal movement information during a kick count session, the kick count session being a period for recording a preset number of fetal movements;
causing the device to display icons on the display screen during the kick count session, each icon corresponding to one of the perceived fetal movements, wherein each of the icons is in the form of a footprint;
recording a heartbeat sound into a data storage of the device;
causing the device to play the heartbeat sound;
upon completion of the kick count session, causing the device to display sequentially the icons on the display screen to perform an animation of feet walking; and
causing the device to play the heartbeat sound during the animation,
whereby the user is able to count the perceived fetal movements recorded during the kick count session by counting the icons displayed during the kick count session.
